(19) United States
(12) Patent Application Publication
(10) Pub. No.: US 2005/0031510 A1
(43) Pub. Date: Feb. 10, 2005

(54) Sterilizing apparatus and sterilizing method

(75) Inventors: Hitoshi Hasegawa, Yokohama-shi (JP); Eiri Suzuki, Sagamihara-shi (JP); Hisashi Kuroshima, Hachioji-shi (JP); Toshiaki Noguchi, Tachikawa-shi (JP)

Correspondence Address: OSTROLENK FABER GERB & SOFFEN, 1180 AVENUE OF THE AMERICAS, NEW YORK, NY, 100368403, US

(73) Assignee: Olympus Corporation

(21) Appl. No.: 10/935,837

(22) Filed: Sep. 8, 2004

Related U.S. Application Data
(63) Continuation of application No. PCT/JP02/09059, filed on Sep. 5, 2002.

(30) Foreign Application Priority Data
Mar. 8, 2002 (JP) 2002-064140

(51) Int. Cl. A61L 2/07

(52) U.S. Cl. 422/295; 422/26

(57) ABSTRACT

A sterilizing apparatus includes a chamber which houses an object to be sterilized so as to sterilize the object using high pressure and temperature steam, an opening/closing section which opens and closes the chamber, and an outflow inhibiting mechanism which operates when the opening/closing section is opened, to inhibit outflow of the steam from the chamber to an exterior in connection with an operation for opening the opening/closing section.

### CROSS-REFERENCE TO RELATED APPLICATIONS

This is a Continuation Application of PCT Application No. PCT/JP02/09059, filed Sep. 5, 2002, which was published under PCT Article 21(2) in Japanese.

This application is based upon and claims the benefit of priority from prior Japanese Patent Application No. 2002-064140, filed Mar. 8, 2002, the entire contents of which are incorporated herein by reference.

### BACKGROUND OF THE INVENTION

1. Field of the Invention

The present invention relates to a sterilizing apparatus comprising a chamber which houses an object to be sterilized so as to sterilize the object using high pressure and temperature steam.

2. Description of the Related Art

Jpn. Pat. Appln. KOKAI Publication No. 11-56977 discloses a high pressure steam sterilizing apparatus in which a heat exchanger plate is provided on an inner surface of a door of a chamber via a spacer to constitute a heating medium channel and in which a heating medium is allowed to flow through the heating medium channel via a heating medium supply port and a heating medium exhaust port to adjust the temperature of the heat exchanger plate to an appropriate value in accordance with an operation, thus preventing operators from being burned.

Further, Jpn. Pat. Appln. KOKAI Publication No. 10-225506 discloses a high pressure steam sterilizing apparatus in which in addition to a sterilizing heater that generates steam during a drying step, a drying heater that dries an object to be sterilized is provided and in which simultaneously with the drying of the object by the drying heater, cooling means cools a part of the apparatus main body to promote evaporation of moisture from the object.

Furthermore, with the conventional high pressure steam sterilizing apparatuses, a user installs the sterilized object in the chamber via a large opening formed in the changer, before executing a sterilizing process.

### BRIEF SUMMARY OF THE INVENTION

According to the first aspect of the present invention, there is provided a sterilizing apparatus comprising:

a chamber which houses an object to be sterilized so as to sterilize the object using high pressure and temperature steam;

an opening/closing section which opens and closes the chamber; and

an outflow inhibiting mechanism which operates when the opening/closing section is opened, to inhibit outflow of the steam from the chamber to an exterior, in connection with an operation for opening the opening/closing section.

According to the second aspect of the present invention, there is provided a sterilizing method comprising:

a step of setting an object to be sterilized in a chamber and closing a opening/closing section provided in the chamber;

a step of executing a sterilizing process by supplying high temperature and pressure steam to the object set in the chamber;

a step of opening the opening/closing section after the sterilizing step has been finished, to take out the object; and

a step of inhibiting outflow of the steam from the chamber to an exterior in connection with an opening operation of the closing section.

### BRIEF DESCRIPTION OF THE SEVERAL VIEWS OF THE DRAWING

FIG. 1 is a view showing the configuration of a high pressure steam sterilizing apparatus according to a first embodiment of the present invention.

FIG. 2 is a block diagram showing the general configuration of the sterilizing apparatus.

FIG. 3 is a flow chart illustrating the procedure of a high pressure steam sterilizing process executed by the sterilizing apparatus.

FIG. 4 is a view illustrating means for reducing the temperature of a chamber opening.

FIG. 5 is a chart showing how the pressure in the chamber varies in accordance with the progress of a series of sterilizing steps as well as timings used to turn on and off or open and close components.

FIG. 6 is a view showing the appearance of a sterilizing apparatus 201 according to a second embodiment of the present invention.

FIG. 7 is a sectional view showing that in the sterilizing apparatus 201 according to the second embodiment, a door 204 is closed.

FIG. 8 is a sectional view showing that in the sterilizing apparatus 201 according to the second embodiment, the door 204 is open.

FIG. 9 is a view illustrating a variation of the second embodiment.

FIG. 10 is a view illustrating another variation of the second embodiment.

### DETAILED DESCRIPTION OF THE INVENTION

Embodiments of the present invention will be described below in detail with reference to the drawings.

(First Embodiment)

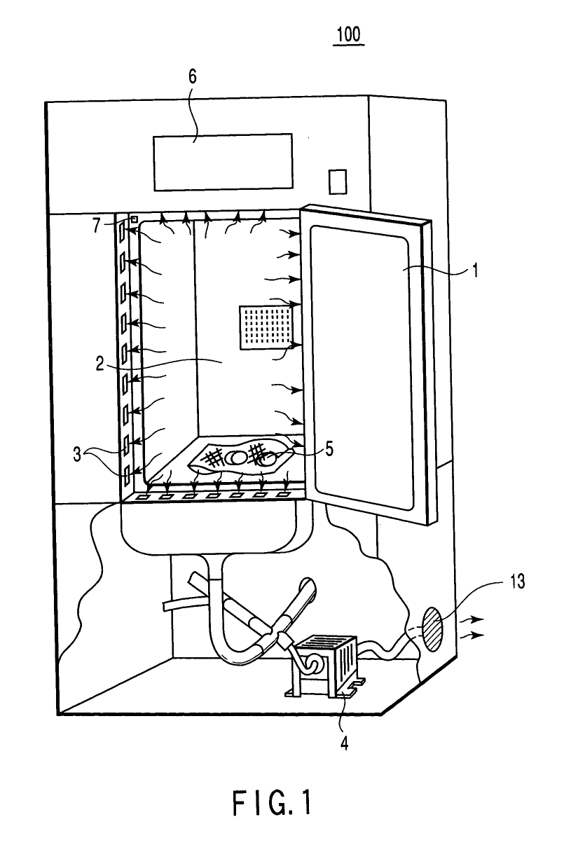

FIG. 1 is a view showing the configuration of a high pressure steam sterilizing apparatus (hereinafter referred to as a sterilizing apparatus) according to a first embodiment of the present invention. A chamber 2 is provided in an almost central portion of the sterilizing apparatus 100. The chamber 2 is used to house an object 5 to be sterilized so as to sterilize it using high pressure and temperature steam. A door (an opening/closing section) 1 is provided to close an opening in the chamber 2. A door sensor 7 senses when the door 1 is closed. A sterilizing process is executed by an operator by operating an operation panel 6 to given an instruction to the apparatus.

Further, a suction duct (outflow inhibiting mechanism) 3 is provided at an opening entry (in the vicinity of a periphery) of the chamber 2 to suck high temperature steam or gas. The suction duct 3 is connected to a suction pump 4 inside the apparatus. Reference numeral 13 denotes an exhaust duct.

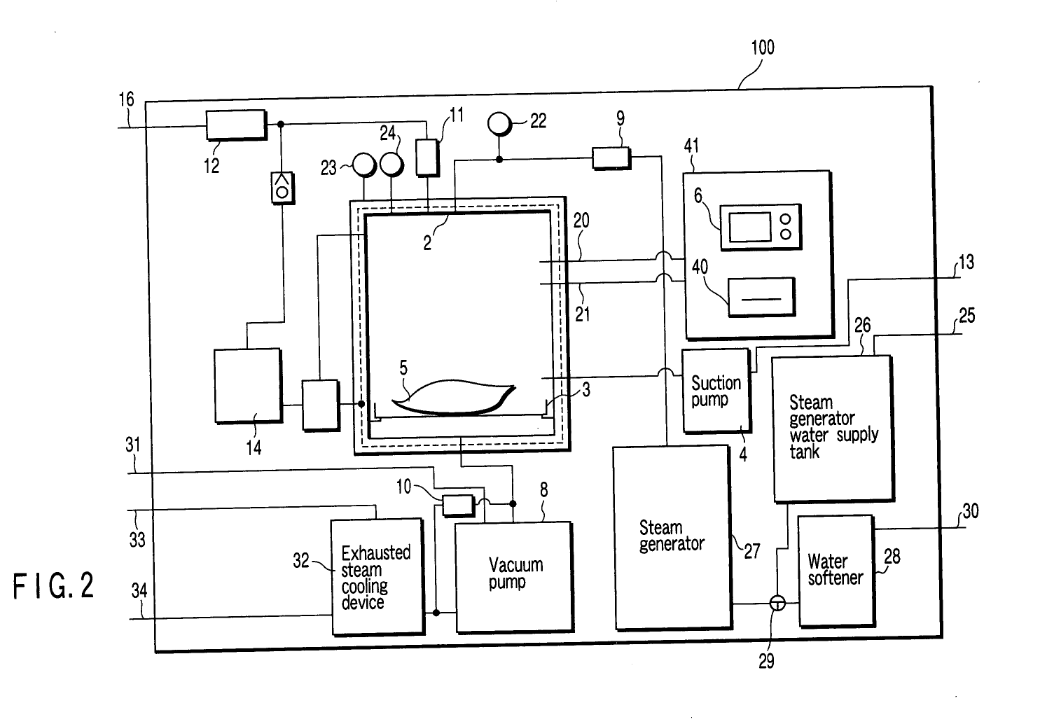

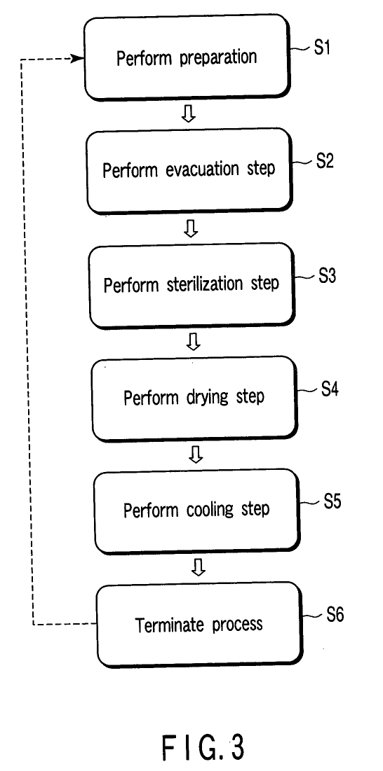

FIG. 2 is a block diagram showing the general configuration of the sterilizing apparatus 100. FIG. 3 is a flow chart illustrating the procedure of a high pressure steam sterilizing process executed by the sterilizing apparatus 100. With reference to FIGS. 1 to 3, description will be given of the high pressure steam sterilizing process according to the present invention. First, as a preparatory step (step S1), an operator opens the door 1, sets the object 5 contained in a sterilizing pack or the like, and closes the door 1. The operator confirms that the door 1 is closed and then pushes a switch on the operation panel 6 to start a sterilizing process. When the door sensor 7 confirms that the door 1 is closed, the door 1 of the apparatus is locked. Then, a series of sterilizing steps described below are started.

First, an evacuation step (step S2) is executed in which a vacuum pump 8 evacuates air from the chamber 2 to reduce the pressure in the chamber 2. Once the pressure in the chamber 2 decreases to a preset value (for example, −0.09 MPa), the vacuum pump 8 is stopped. Then, a steam supply valve 9 is opened to introduce high pressure steam generated by a steam generator 27 into the chamber 2. Thus, a sterilizing step (step S3) is started. Steam is fed into the chamber 2 until the temperature in the chamber reaches a preset sterilizing temperature (for example, 135° C.). To improve efficiency, the vacuum evacuation and the steam supply may be alternated.

Once the temperature in the chamber 2 reaches the set sterilizing temperature, the interior of the chamber 2 is kept at this temperature for a preset sterilizing time (for example, 5 minutes). When the sterilizing time has passed, a steam discharge valve 10 is opened to discharge the high pressure steam from the chamber 2 to its exterior. Then, an exhausted steam cooling device 32 uses cooling water supplied through a cooling water supply port 33 to cool and convert the discharged high pressure steam into water. The water is then drained from a drain exhaust port 34.

After the steam has been discharged, a drying step (step S4) is executed in which the vacuum pump 8 reduces the pressure in the chamber 2. This reduces the pressure in the chamber 2 while maintaining the high temperature. Consequently, water droplets are rapidly vaporized and dried.

When a preset drying time (for example, 10 minutes) has passed, the vacuum pump 8 is stopped. Then, an intake valve 11 is opened to return the pressure in the chamber 2 to atmospheric pressure. To prevent the interior of the chamber 2 from being contaminated by indigenous bacteria in the air, a filter called a sterile filter 12 and having a filter mesh of at most 0.2 microns is attached to an air intake port 16 of the intake valve 11.

Once the pressure in the chamber 2 has returned to atmospheric pressure, the door 1 is unlocked. The operator confirms that the door 1 has been unlocked and then opens the door 1. At this time, the door sensor 7 senses the opening to start the suction pump 4 to execute a cooling step (step S5). Further, since the temperature in the chamber 2 is high, remaining vapor or steam is sucked through the suction duct 3 in the chamber opening. The vapor passes through the suction pump 4 and is then discharged from the exhaust duct 13, located in the rear of a side of the apparatus, to the exterior of the apparatus.

When a preset specified time has passed or the temperature of the atmosphere, measured by a temperature sensor 20 provided in the chamber 2 or near the opening in the chamber 2, becomes equal to or lower than a set temperature, the suction pump 4 is stopped. This minimizes a decrease in the temperature in the chamber 2 in order to maintain a preheating effect for the next sterilizing operation. Thus, a series of sterilizing steps are completed (step S6). The operator can then set the next object to be sterilized in the chamber 2.

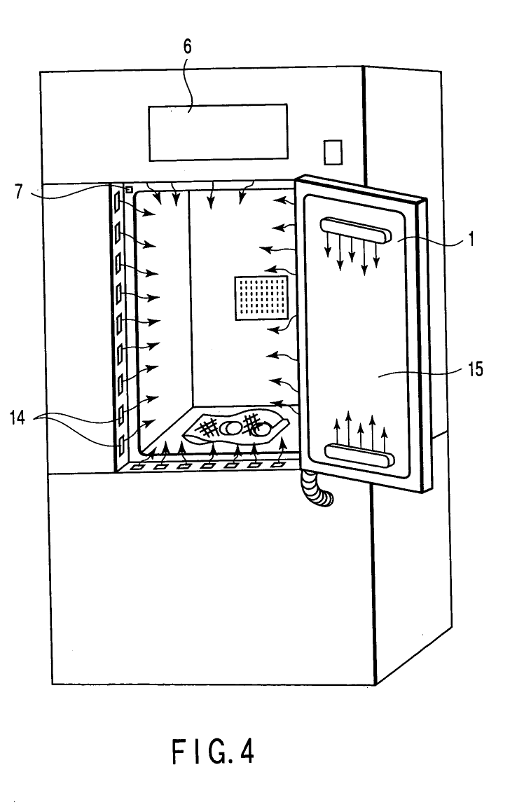

As means for reducing the temperature in the chamber opening, the door sensor 7 may sense when the door 1 is opened as shown in FIG. 4, so that cooling air may shoot from a cooling duct 14 provided around the periphery of the chamber opening or inside the door 1. Alternatively, as shown in FIG. 4, heat-sensitive paint 15 or a seal like object which has its color changed by the temperature may be attached to the inside of the door 1 or to the chamber opening so as to allow the operator to visually confirm that the temperature has sufficiently decreased.

In FIG. 2, reference numerals 21, 22, 23, and 24 denote pressure gauges. The operation panel, together with a recording device 40, constitutes a control box 41. A steam generator water supply tank 26 connected to a manual water supply port 25 and a water softener 28 connected to a steam water supply port 30 are connected the steam generator 27 via a change-over valve 29.

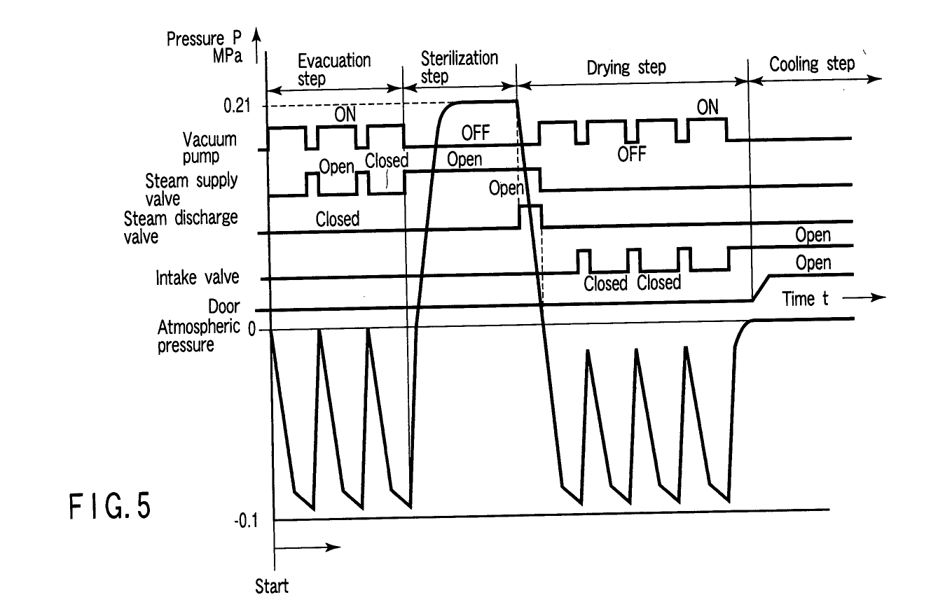

A time chart in FIG. 5 shows how the pressure in the chamber 2 varies in accordance with the progress of the series of sterilizing steps as well as timings used to turn on and off or open and close the components. During an ON period of the vacuum pump 8, the pump 8 is repeatedly turned on and off. The timings for turn on and off correspond to the waveform of atmospheric pressure.

According to the first embodiment, in a high pressure steam sterilizing apparatus that sterilizes an object using high temperature steam or gas, after sterilization, the suction pump 3 or a fan is operated at almost the same time when the door 1, which closes the chamber 2, is opened. Further, the suction duct 3, provided around the periphery of the opening in the chamber 2, sucks and discharges high temperature steam and gas to the exterior of the apparatus. This improves operability.

(Second Embodiment)

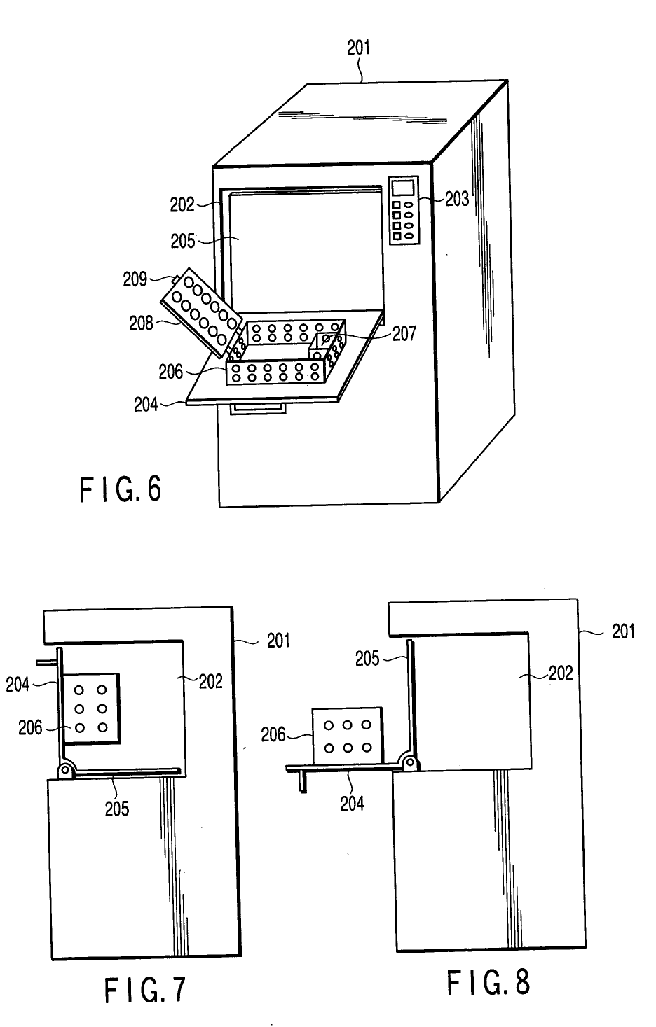

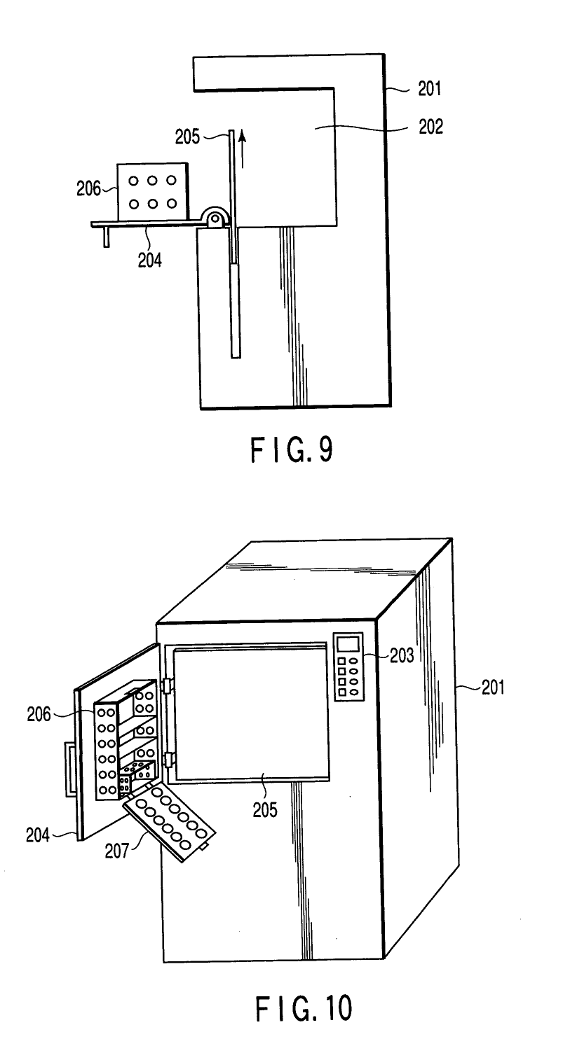

Now, a second embodiment will be described with reference to FIGS. 6 to 8. FIG. 6 shows the appearance of a sterilizing apparatus 201 according to the second embodiment. When an object is housed in a chamber 202 of the sterilizing apparatus 201, a sterilizing process is executed using high pressure steam. Further, the sterilizing apparatus 201 is provided with an operation panel 203. The sterilizing apparatus 201 is operated by operating the operation panel 203. The sterilizing apparatus 201 is provided with a door 204 so as to open and close the chamber 202. A screen 205 is formed integrally with the door 204. When the door 204 is opened, the screen (outflow inhibiting mechanism) 205 shields a chamber opening.

Further, the door 204 is opened until its horizontal position. When the door 204 is opened, a housing case 206 appears which is fixed to the inside of the door 204 to house an object. The housing case 206 is provided with an indicator used to check whether or not the object has been reliably sterilized and an indicator installation space 207 used to install the indicator or a test pack.

Moreover, the housing case 206 is provided with a opening/closing cover 208 with a lock portion 209. Further, the housing case 206, its opening/closing cover 208, and the indicator installation space 207 are provided with a large number of vent holes to allow steam to permeate into the housing case 296 during a sterilizing process.

FIG. 7 is a sectional view showing the sterilizing apparatus 201 according to the present embodiment, in which the door 204 is closed. FIG. 8 is a sectional view showing the sterilizing apparatus 201, in which the door 204 is open. The door 204 and the screen 205 are formed integrally with each other so as to form a right angle between them. Thus, in the state shown in FIG. 7, when the door 204 is opened until its horizontal position, the screen 205 stands up to its vertical position to close the opening in the chamber 202 (FIG. 8).

Now, description will be given of the operation of the second embodiment of the present invention. When an object is to be sterilized, the door 204 is first opened to draw out the housing case 206. At this time, the screen 205, integrated with the door 204, closes the opening in the chamber 202. It is thus possible to avoid allowing steam or vapor in the chamber 202 to leak out to prevent the user's operations or reducing the temperature in the chamber 202.

Then, the door 204 is opened until its horizontal position to draw out the housing case 206. Then, the opening/closing cover 208 is opened and the object is installed in the housing case 206. Further, at this time, the indicator, used to check whether or not the object has been reliably sterilized, and a test pack are also installed in the indicator installation space 207. Then, finally, the opening/closing cover 208 is closed and is locked using the lock portion 209.

In this manner, the object can be installed from the exterior of the chamber 202. This eliminates the need to access the interior of the chamber 202 to allow the object to be easily installed. Consequently, the user can perform operations more easily and efficiently. Then, after the object has been housed in the housing case 206, the door 204 is closed to place the housing case 206 in the chamber 202 to load the object into the chamber 202.

After the door 204 has been closed, the operation panel 203 is operated to execute a sterilizing process. When the sterilizing process is started in the sterilizing apparatus 201, sterilizing high temperature vapor is supplied into the chamber 202. Then, the vapor permeates into the housing case 206 through the vent holes in the housing case 206 to come into contact with the object for sterilization.

Once a predetermined sterilizing process has been finished, the operation panel 203 shows that the process has been finished. The user confirms this display and then opens the door 204 to take out the object. In this case, the door 204 and the screen 205 are formed integrally with each other so as to have a right angle between them. Accordingly, when the door 204 is opened until the horizontal position, the screen 205 stands up to its vertical position to close the opening in the chamber 202. This prevents the steam in the chamber 202 from leaking out.

This configuration can prevent the temperature of the chamber 202 from decreasing. Thus, during subsequent sterilizing processes, it is possible to shorten a preheating step of preheating the chamber 202. Then, the housing case 206 is drawn out toward the user by opening the door 204. The lock portion 209 is then released to open the opening/closing door 208. The sterilized object is then taken out.

In this manner, the sterilized object can be loaded into and taken out of the chamber 202 by opening and closing the door 204. Accordingly, the operator can easily perform a sterilizing operation without accessing the interior of the chamber 202.

In the above embodiment, the screen 205 is integrated with the door 204. However, the screen 205 and the door 204 may be formed separately so that when the door 204 is opened, a motor is used to automatically raise the screen 205 from the bottom of the chamber 202. Of course, conversely, the door 204 may be lowered from above.

Alternatively, the door 204 may be opened and closed in a lateral direction as shown in FIG. 10 rather than toward the user as in the case of the above embodiment. Further, the housing case 206 may have a mesh structure rather than being provided with the large number of vent holes as in the case of the above embodiment.

According to the second embodiment, the object to be sterilized is housed and taken out by opening and closing the chamber opening/closing mechanism of the sterilizing apparatus. Consequently, the user need not access the interior of the chamber and can thus perform operations more easily and efficiently. Further, when the opening/closing means is opened, the opening in the chamber is closed. This prevents a decrease in the temperature of the chamber 202 to shorten the preheating step.

According to the present invention, after sterilization, when the door that closes the chamber is opened, high temperature steam or gas remaining in the chamber is sucked through the suction duct and discharged to the exterior of the apparatus. This improves operability.

Further, according to the present invention, the sterilized object is housed and taken out by opening and closing the chamber opening/closing mechanism of the sterilizing apparatus. Consequently, the user need not access the interior of the chamber and can thus perform operations more easily and efficiently. Furthermore, when the opening/closing means is opened, the opening in the chamber is closed. This serves to maintain the temperature of the chamber to shorten the preheating step.

1. A sterilizing apparatus comprising:
a chamber which houses an object to be sterilized so as to sterilize the object using high pressure and temperature steam;
an opening/closing section which opens and closes the chamber; and
an outflow inhibiting mechanism which operates when the opening/closing section is opened, to inhibit outflow of the steam from the chamber to an exterior, in connection with an operation for opening the opening/closing section.

2. The sterilizing apparatus according to claim 1, wherein the outflow inhibiting mechanism includes a sucking section provided in the vicinity of a periphery of an opening through which the object is housed in or taken out of the chamber, and sucks the steam via the sucking section in connection with the operation for opening the opening/closing section.

3. The sterilizing apparatus according to claim 1, wherein the outflow inhibiting mechanism includes a shielding member which shields the opening through which the object is housed in or taken out of the chamber, in connection with the operation for opening the opening/closing section.

4. The sterilizing apparatus according to claim 3, wherein the shielding member is integrated with the opening/closing section and moves to a position where the shielding member completely shields the opening when the opening/closing section is opened until the opening/closing section reaches an open position outside the apparatus.

5. The sterilizing apparatus according to claim 4, wherein a housing section in which the object is housed is provided behind the opening/closing section in a state of being closed, and when the opening/closing section is opened until the opening/closing section reaches the open position outside the apparatus, the housing section is placed at a position where the housing section can house the object.

6. The sterilizing apparatus according to claim 5, wherein the housing section is provided with an indicator installation space in which an indicator used to check whether or not the object has been reliably sterilized and others are installed.

7. The sterilizing apparatus according to claim 6, wherein the indicator installation space is provided with a plurality of vent holes into which steam permeates during a sterilizing process.

8. The sterilizing apparatus according to claim 6, wherein the indicator installation space has a mesh structure into which steam permeates during a sterilizing process.

9. The sterilizing apparatus according to claim 3, wherein the shielding member is provided separately from the opening/closing section and moves to a position where the shielding member completely shields the opening when the opening/closing section is opened until the opening/closing section reaches an open position outside the apparatus.

10. A sterilizing method comprising: a step of setting an object to be sterilized in a chamber and closing a opening/closing section provided in the chamber;
a step of executing a sterilizing process by supplying high temperature and pressure steam to the object set in the chamber;
a step of opening the opening/closing section after the sterilizing step has been finished, to take out the object; and
a step of inhibiting outflow of the steam from the chamber to an exterior in connection with an opening operation of the closing section.
